(19) United States
(12) Patent Application Publication
(10) Pub. No.: US 2024/0293065 A1
(43) Pub. Date: Sep. 5, 2024

(54) SYSTEMS AND METHODS FOR DISPOSABLE SENSORS

(71) Applicant: GE Precision Healthcare LLC, Waukesha, WI (US)

(72) Inventors: Christopher Gabel, Waukesha, WI (US); Yanju Wang, Hartland, WI (US); Sean Walgrave, Oconomowoc, WI (US); Christopher Ian Johnson, Macclesfield (GB); Robert Brendan Smith, Beeston (GB); Daniel Garcia, Pewaukee, WI (US)

(21) Appl. No.: 18/574,873

(22) PCT Filed: Jun. 29, 2022
(86) PCT No.: PCT/US2022/035526

§ 371 Date: Dec. 28, 2023

Related U.S. Application Data
(60) Provisional application No. 63/216,267, filed on Jun. 29, 2021.

(51) Int. Cl. A61B 5/266; A61B 5/00; A61B 5/257; A61B 5/265; A61B 5/28; A61B 5/291; A61B 5/296

(52) U.S. Cl. CPC A61B 5/266; A61B 5/257; A61B 5/265; A61B 5/68335; A61B 5/28; A61B 5/291; A61B 5/296; A61B 2562/0215; A61B 2562/164; A61B 2562/168; A61B 2562/227

(57) ABSTRACT

Various systems and methods for managing disposable sensors and electrode gel are disclosed. According to an embodiment, a sensor assembly configured to be attached to a patient includes one or more electrode modules. Each of the one or more electrode modules includes an electrode, electrode gel disposed in a storage location on the electrode module, wherein electrode gel is not in contact with the electrode in the storage location, and a deformable cap configured to move the electrode gel from the storage location into contact with the electrode in response to a deformation of the deformable cap.

### CROSS-REFERENCE TO RELATED APPLICATIONS

This is a national stage application under 35 U.S.C. § 371(c) of PCT Application No. PCT/US2022/035526, filed on Jun. 29, 2022, which claims priority to U.S. Provisional Patent Application No. 63/216,267, filed on Jun. 29, 2021, the disclosures of which are incorporated herein by reference in their entireties.

### BACKGROUND

The subject matter disclosed herein relates generally to a sensor assembly that is configured to be attached to a patient including one or more electrode modules. Each of the electrode modules includes an electrode, a deformable cap, and electrode gel disposed in a storage location on the sensor assembly. The electrode gel is not in contact with the electrode while the electrode gel is in the storage location. A method of using the sensor assembly including the electrode, the deformable cap, and the electrode gel is also disclosed herein.

When using sensor assemblies that include one or more electrodes for the purpose of either detecting electrical signals from a patient or transmitting electrical signals to the patient, it is common to use electrode gel to improve the coupling of the electrode to the patient. The electrode gel acts as an electrical bridge between each electrode and the patient's skin to enable the efficient transfer of electrical signals between the patient's skin and each respective electrode. The electrode gel, may, for instance, be an electrolyte gel including compounds such as sodium chloride (NaCl) or potassium chloride (KCl). The addition of salt compounds such as sodium chloride (NaCl) or potassium chloride (KCl) in the electrode gel facilitates the transmission of electrical signals either from the patient to the electrode or from the electrode to the patient. With some conventional solutions, it is common to have a clinician manually apply the electrode gel to each electrode prior to attaching each electrode to the patient. However, the application of the electrode gel is cumbersome and potentially messy for the clinician. In addition, manually applying the electrode gel to the electrodes requires the clinician to have an adequate supply of electrode gel on-hand at all times to ensure that there is gel available for each new patient.

Due to the aforementioned challenges with the manual application of electrode gel, some conventional solutions include a quantity of electrode gel pre-applied to the sensor assembly. The electrode gel is typically in a stored in contact with the electrode on the sensor assembly. According to conventional solutions, the clinician would typically need to remove a film or cover on top of the electrode gel disposed on the electrode before placing the electrode, including the pre-applied electrode gel, onto the skin of the patient. While using sensor assemblies with pre-applied electrode gel is more convenient for the clinician than manually applying electrode gel from a separate container, conventional solutions still leave much to be desired in terms of long-term storage of the sensor assembly (with the pre-applied electrode gel).

As discussed above, the electrode gel typically includes salt compounds such as sodium chloride (NaCl) or potassium chloride (KCl). Storing the sensor assembly with the pre-applied electrode gel according to conventional techniques has been shown to create significant issues regarding the reliability of the electrodes. The electrode gel is known to be corrosive to the materials commonly used for the electrodes. This interaction between the electrode gel and the electrode results in electrode degradation and an unacceptable rate of failures for sensor assemblies with electrode gel pre-applied to each electrode particularly when the sensor assemblies are stored with the pre-applied electrode gel.

For at least the reasons discussed hereinabove, there is the need for an improved sensor assembly including a storage location for electrode gel and an improved method of using a sensor assembly including the storage location for the electrode gel.

### BRIEF DESCRIPTION

In an aspect, a sensor assembly configured to be attached to a patient for at least one of detecting an electrical signal from the patient or providing an electrical signal to the patient includes one or more electrode modules, wherein each of the one or more electrode modules includes an electrode, electrode gel disposed in a storage location on the electrode module, wherein the electrode gel is not in contact with the electrode in the storage location, and a deformable cap configured to move the electrode gel from the storage location into contact with the electrode in response to a deformation of the deformable cap.

In an aspect, a method of using a sensor assembly including one or more electrode modules, wherein each of the one or more electrode modules includes an electrode and a deformable cap. The method includes pressing the deformable cap into a deformed position and transferring electrode gel from a storage location into contact with the electrode in response to said pressing the deformable cap.

### BRIEF DESCRIPTION OF THE DRAWINGS

The inventive subject matter described herein will be better understood from reading the following description of non-limiting embodiments, with reference to the attached drawings, wherein below.

FIG. 1 shows a schematic diagram of a sensor assembly in accordance with an embodiment;

FIG. 2 shows a schematic diagram of a sensor assembly in accordance with an embodiment;

FIG. 3A shows a top view of an electrode module of a sensor assembly in accordance with an embodiment;

FIG. 3B shows a sectional view of the electrode module of FIG. 3A along line A-A′ in accordance with an embodiment;

FIG. 3C shows a sectional view of the electrode module of FIG. 3A along line B-B′ in accordance with an embodiment;

FIG. 3D shows a sectional view of the electrode module of FIG. 3A along line A-A′ with the deformable cap in a deformed position in accordance with an embodiment;

FIG. 4 shows a flow chart of a method in accordance with an embodiment;

FIG. 5A shows a top view of an electrode module of a sensor assembly in accordance with an embodiment;

FIG. 5B shows a sectional view of the electrode module of FIG. 5A along line C-C′ in accordance with an embodiment;

FIG. 6A shows a top view of an electrode module of a sensor assembly in accordance with an embodiment;

FIG. 6B shows a sectional view of the electrode module of FIG. 6A along line D-D′ in accordance with an embodiment;

FIG. 6C shows a sectional view of the electrode module of FIG. 6A along line D-D′ in accordance with an embodiment;

FIG. 7A shows a top view of an electrode module of a sensor assembly in accordance with an embodiment;

FIG. 7B shows a sectional view of the electrode module of FIG. 7A along line E-E′ in accordance with an embodiment;

FIG. 7C shows a sectional view of the electrode module of FIG. 7A along line E-E′ in accordance with an embodiment;

FIG. 8A shows a top view of an electrode module of a sensor assembly in accordance with an embodiment;

FIG. 8B shows a sectional view of the electrode module of FIG. 8A along line F-F′ in accordance with an embodiment;

FIG. 8C shows a sectional view of the electrode module of FIG. 8A along line F-F′ in accordance with an embodiment.

FIG. 9A shows a top view of an electrode module of a sensor assembly in accordance with an embodiment;

FIG. 9B shows a sectional view of the electrode module shown in FIG. 9A along line G-G′ in accordance with an embodiment; and

FIG. 9C is a sectional view of the electrode module shown in FIG. 9A along line G-G′ in accordance with an embodiment.

### DETAILED DESCRIPTION

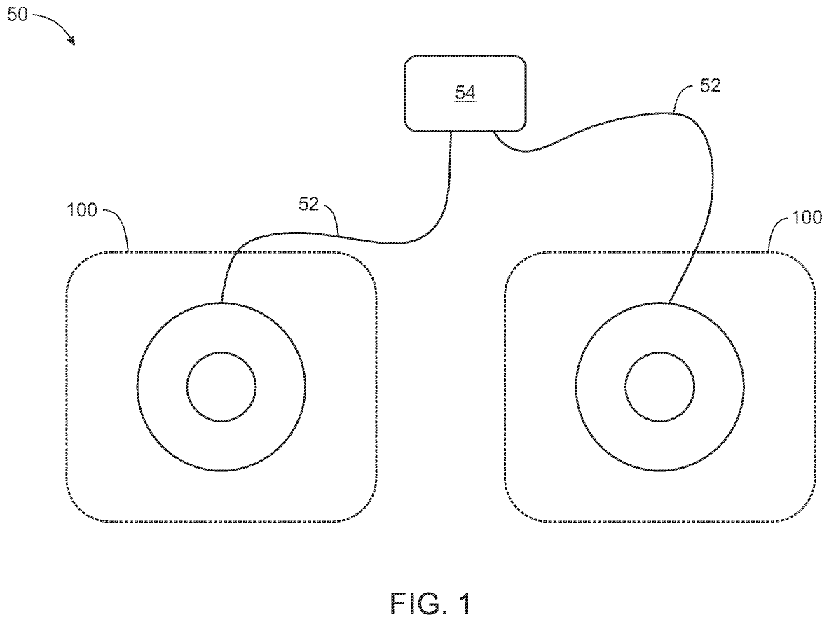

FIG. 1 illustrates a sensor assembly 50 in accordance with an embodiment. The sensor assembly 50 is configured to be attached to the skin of a patient in order to detect one or more electrical signals from the patient and/or to provide one or more electrical signals to the patient. The sensor assembly 50 includes an electrode module 100. The embodiment shown in FIG. 1 is depicted with two electrode modules 100. Each electrode module 100 includes an electrode and is configured to be attached to the skin of a patient to establish an electrical connection between the associated electrode and the skin of the patient. Details about the electrode module 100 will be discussed hereinafter.

In the embodiment shown in FIG. 1, each electrode module 100 is connected to a junction 54 via a wire 52. According to other embodiments, each electrode module may be wirelessly connected to the junction 54 via wireless techniques such as Wi-Fi, Bluetooth, Near-Field Communication (NFC) techniques, or according to any other wireless communication technique. The junction 54 may be configured to be in electronic communication with a processor of a separate sensor assembly (not shown). The junction 54 may be configured to be in electronic communication with the separate sensor assembly via either a wired connection or via wireless communication techniques such as Wi-Fi, Bluetooth, Near-Field Communication (NFC) techniques, or according to any other wireless communication technique. According to some embodiments, the junction 54 may further include a plug to establish a wired connection with a separate sensor assembly (not shown) such as a monitoring system.

According to an embodiment, the sensor assembly 50 may, for example, be a sensor configured to be used with a medical sensor assembly. For example, the sensor assembly 50 may be a sensor that is configured to be used with a medical sensor assembly such as an electrocardiograph (ECG) monitoring sensor assembly, an electroencephalograph (EEG) monitoring sensor assembly, an electromyography (EMG) monitoring sensor assembly, a patient motion detecting sensor assembly, or any other type of patient monitoring sensor assembly that is configured to be used by electrically connecting one or more electrodes to the skin of the patient. According to other embodiments, the sensor assembly 50 may be configured to be used with a therapy sensor assembly or a stimulation sensor assembly. For example, the sensor assembly 50 may be configured to be used with a neuromuscular stimulation sensor assembly or any other type of sensor assembly that is configured to be used by electrically connecting one or more electrodes to the skin of the patient.

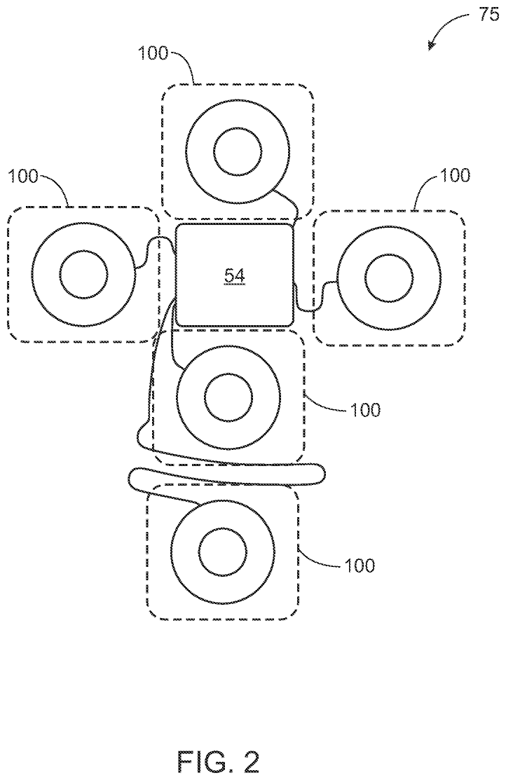

FIG. 2 illustrates a top view of a sensor assembly 75 in accordance with an embodiment with five electrode modules. Each electrode module 100 shown on FIG. 2 is connected to the junction box 54. The sensor assembly 75 shown in FIG. 2 may be a maternal/fetal monitoring sensor and that is configured to be attached to the abdomen of a pregnant patient. Each of the five electrode modules 100 is configured to be attached to the skin of the patient. The sensor assembly 75 is configured to receive signals from the mother and fetus related to cardiac performance and/or uterine activity. According to an exemplary embodiment, the sensor assembly 75 may be configured to detect signals used to determine fetal heart rate, maternal heart rate, and uterine activity. The sensor assembly 75 is configured to be in electronic communication with a monitoring system, via either wired or wireless techniques. According to an exemplary embodiment, the junction box 54 of the sensor assembly 75 may include a wireless transmitter configured to be in electronic communication with the monitoring system. The monitoring system includes a processor that receives the signals from the sensor assembly 75 and converts the signals into a format appropriate for display on a display sensor assembly of the monitoring system.

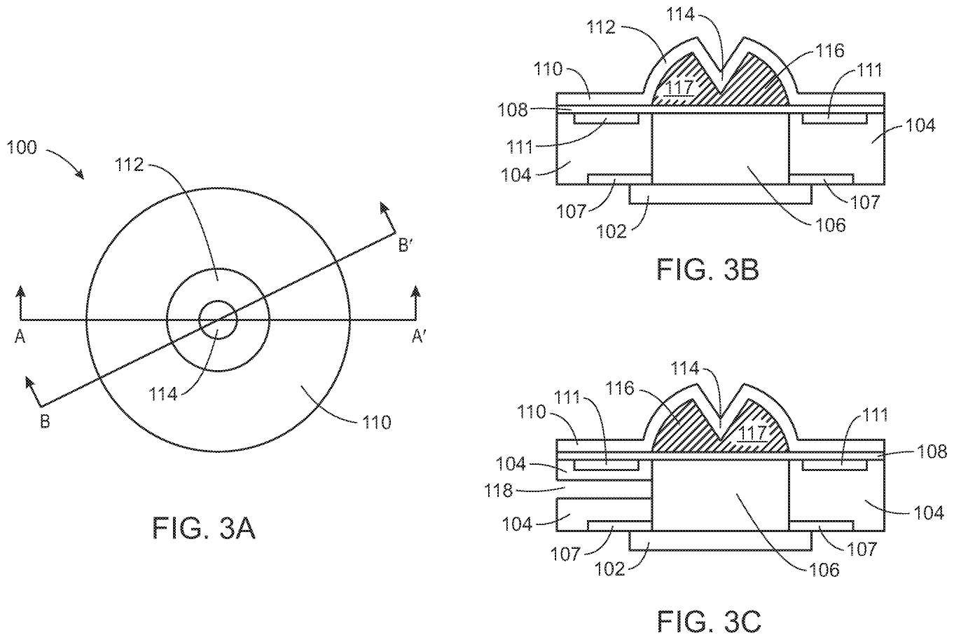

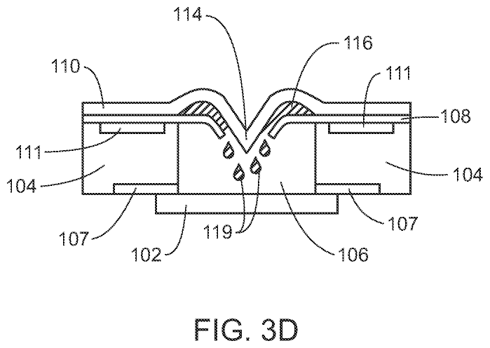

FIG. 3A illustrates a top view of an electrode module 100 of a sensor assembly in accordance with an embodiment. FIG. 3B illustrates a sectional view of the electrode module 100 shown in FIG. 3A along line A-A′ in accordance with an embodiment. FIG. 3C illustrates a sectional view of the electrode module 100 of FIG. 3A along line B-B′ in accordance with an embodiment. FIG. 3D illustrates a sectional view of the electrode module 100 of FIG. 3A along line A-A′ with the deformable cap in a deformed position in accordance with an embodiment.

The electrode module 100 includes an electrode 102, a conformable wall 104, a gel-retaining structure 106, a film 108, and a deformable cap 110. According to embodiments, the gel-retaining structure 106 may be configured to retain a liquid in contact with the electrode 102. This may be advantageous for embodiments where the electrode gel 117 is a liquid or has a viscosity that is close to that of a liquid. The electrode 102 may be formed of any conductive material, such as a conductive metal or a conductive metal alloy. However, according to exemplary embodiments, the electrode 102 may include a conductive metal alloy such as silver (Ag), silver chloride (AgCl), a silver/silver chloride mixture, stainless steel, copper, aluminum, or any other electrically conductive metals or alloys, or conductive carbon. The electrode 102 is configured to either provide an electrical signal from a signal generator (not shown) or to receive an electrical signal from a patient (not shown). According to some embodiments, the electrode 102 may be deposited through an additive process, such as via a printing process using a conductive ink.

The conformable wall 104 is formed from a flexible material such as, for example, rubber, closed-cell foam, silicone, or a thermoplastic, such as, for example, polystyrene, polyolefin, polyester, polyurethane, polyamide, polysulfone, polyethersulfone, polycarbonate, or the like, or any other flexible material configured to conform to the patient when the electrode module 100 is positioned on the patient's skin. The conformable wall 104 may be shaped in a generally ring-like shape substantially surrounding the gel-retaining structure 106.

The deformable cap 110 may be constructed from any material that is configured to be deformable in response to pressure applied by a clinician. For example, the deformable cap 110 may be constructed from a plastic, such as a thermoplastic, such as, for example, polystyrene, polyolefin, polyester, polyurethane, polyamide, polysulfone, polyethersulfone, polycarbonate, or the like. The deformable cap 110 may be constructed from any other flexible material according to various embodiments. The material for the deformable cap 110 may selected to have a relatively low melting point, such as below 250 C degrees. The film 108 is a thin layer that provides a barrier between at least a portion of the deformable cap 110 and the gel-retaining structure 106 in the embodiment shown in FIGS. 3A, 3B, 3C, and 3D. According to an embodiment, the film 108 may be attached to the deformable cap 110. According to an exemplary embodiment, the film 108 and the deformable cap 110 may be made of the same material. For example, the film 108 may also be made from a plastic such as polystyrene, polyolefin, or any other plastic polymer. According to other embodiments, the film 108 may be formed from a different material than the deformable cap 110. According to an embodiment, the film 108 may be coated with a release coating on all or a portion of one side to allow for the film 108 to be easily separated from the electrode module 100 after the electrode gel 117 has been moved from the storage location 116 into contact with the electrode 102.

The material used to form the film 108 may be thinner than the material used to form the deformable cap 110. For example, the film 108 may be configured to be relatively easy to puncture with a punch pin. The film 108 may be, for example, between 0.1 mils and 2 mils in thickness, or more specifically between 0.5 mils and 1.5 mils. According to embodiments where the film 108 is attached to the deformable cap 110, such as the embodiment shown in FIGS. 3A, 3B, 3C, and 3D, the film 108 may be attached to the deformable cap 110 using any conventional attachment techniques. For example, the film 108 may be thermo-bonded to the deformable cap 110, the film 108 may be ultrasound-bonded to the deformable cap 110, or the film 108 may be adhesively bonded to the deformable cap 110.

The deformable cap 110 may be shaped to include a raised portion 112 and to define one or more punch pins, such as a punch pin 114 shown in FIGS. 3B, 3C, and 3D. The deformable cap 110 and the film 108 may collectively define a storage location 116 for a quantity of electrode gel 117 as shown in FIGS. 3B and 3C. The storage location 116 may be, for example, a chamber defined by the deformable cap 110 and the film 108 according to an embodiment. The electrode gel 117 is represented with hatching (//) in the figures. The electrode gel 117 may be any type of gel that is configured to conduct electricity. According to embodiments, the electrode gel 117 may include salt compounds such as sodium chloride (NaCl) or potassium chloride (KCl) in order to facilitate the conduction of electricity between the electrode 102 and the skin of a patient (not shown). The electrode gel 117 may include other salts such as, for example, sodium, potassium, magnesium chloride, magnesium acetate, magnesium sulfate, etc., according to various embodiments.

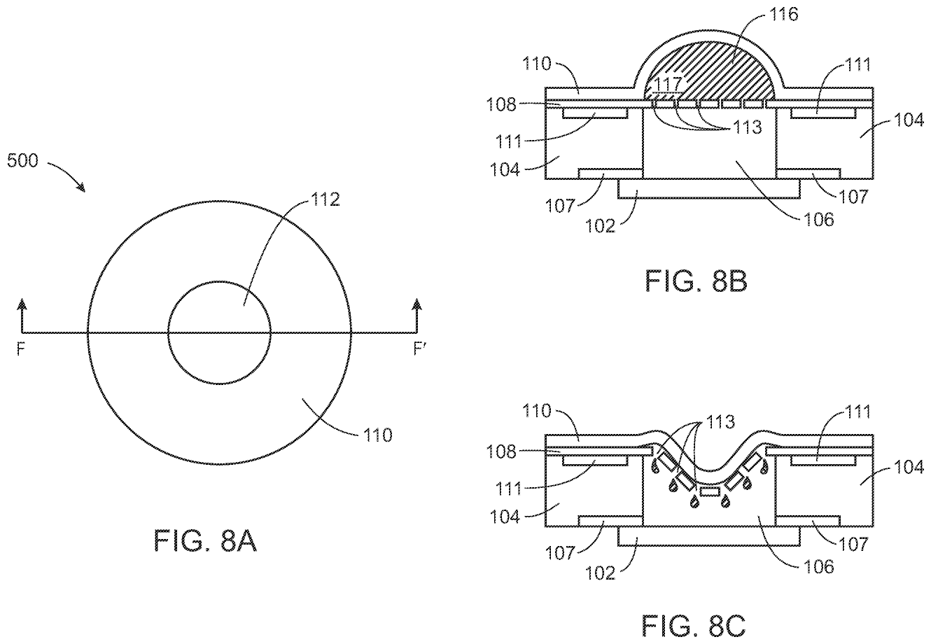

According to the embodiment shown in FIGS. 3B and 3C, the film 108 is configured to keep the electrode gel 117 from contacting the electrode 102 while the film 108 is intact and the electrode gel 117 is in the storage location 116. The film 108 may be configured to be impervious to the electrode gel 117. According to other embodiments, which will be discussed hereinafter, the film 108 may be perforated with a plurality of perforations. The sizes of the perforations may be selected to prevent the transmission of the electrode gel 117 until the application of a threshold amount of pressure. Additional details about the perforations will be discussed hereinafter with respect to FIGS. 8A, 8B, and 8C.

FIG. 3C illustrates a sectional view of the electrode module 100 of FIG. 3A along line B-B′ in accordance with an embodiment. FIG. 3C shows how the conformable wall 104 may be shaped to define a passageway 118. The passageway 118 is configured to provide a path for air to escape. The passageway 118 connects the gel-retaining structure 106 with ambient air surrounding the electrode module 100. Additional details about the passageway 118 will be discussed hereinafter.

According to some embodiments, the conformable wall 104 may be attached to the electrode 102 using a first adhesive 107, such as a pressure sensitive adhesive (PSA). The electrode module 100 includes a second adhesive 111. The second adhesive 111 is located between the film 108 and the conformable wall 104. The second adhesive 111 is configured to be used to attach the electrode module 100 to the skin of the patient after the film 108 and deformable cap 110 have been removed from the electrode module 100. According to an embodiment, a release coating may be applied to one side of the film 108 in order to prevent the film 108 from adhering to the second adhesive 111. FIGS. 3B, 3C, and 3D illustrate how the first adhesive 107 may be positioned to adhere the electrode module 100 to the conformable wall 104. The electrode 102, may be attached to the conformable wall 104 using other techniques, such as adhesive bonding, thermo-bonding, ultrasound-bonding, or any other known attachment technique.

The gel-retaining structure 106 is configured to retain the electrode gel 117 in contact with the electrode 102 after the electrode gel 117 has been moved from the storage location 116 into contact with the electrode 102. The gel-retaining structure 106 may comprise an open-cell sponge, a wire frame sponge, a deformable ring, or any other structure configured to keep electrode gel 117 in contact with the electrode 102.

According to embodiments where the gel-retaining structure 106 is a wire frame sponge, the wire frame sponge may include a plurality of connected metal or plastic wires that collectively form a wire frame, or lattice structure, that is configured to retain the electrode gel 117. For example, according to an embodiment where the gel-retaining structure 106 is an open-cell sponge, the open-cell sponge may be configured to become saturated with the electrode gel 117 after the film 108 has been punctured. According to an embodiment where the gel-retaining structure 106 is a deformable ring, the deformable ring may, for instance, be made of a soft material such as rubber or silicone. The deformable ring may be configured to retain the electrode gel 117 within a central portion of the deformable ring.

FIG. 3D illustrates a sectional view of the sensor assembly of FIG. 3A along line A-A′ with the deformable cap 110 in a deformed position in accordance with an embodiment. FIGS. 3B and 3C both show the deformable cap 110 in an undeformed position. FIG. 3D additionally shows the punch pin 114 piercing the film 108. The piercing or puncturing of the film 108 allows the electrode gel 117 to travel from the storage location 116 to the gel-retaining structure 106. A plurality of gel droplets 119 with hatching are included on FIG. 3D to schematically represent the flow of the electrode gel 117 from the storage location 116 to the gel-retaining structure 106 after the film 108 has been punctured by the punch pin 114.

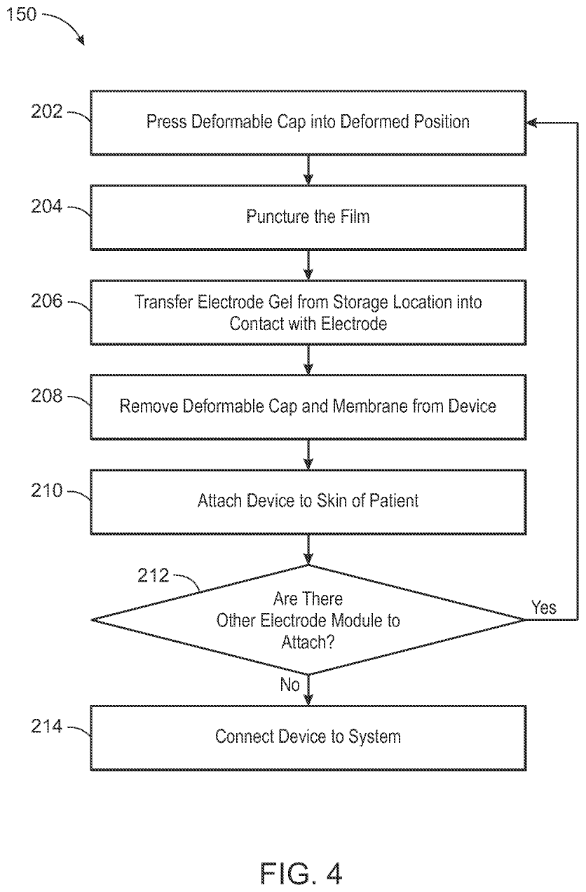

FIG. 4 is a flow chart of a method 150 in accordance with an embodiment. The method 150 may will be described according to an embodiment with electrode modules configured as shown in FIGS. 3A, 3B, 3C, and 3D. But it should be appreciated that the method 150 may be performed with electrode modules configured differently according to other embodiments. The technical effect of the method 150 is the transfer of electrode gel from a storage location on the electrode module into contact with the electrode in order to enhance the electrical conductivity between the patient and the electrode.

The method 150 will be described with respect to FIGS. 3A, 3B, 3C, and 3D. At step 202, pressure is applied to the deformable cap 110. According to an embodiment, a user may press the raised portion 112 of the deformable cap 110 into a deformed position, such as that shown in FIG. 3D. At step 204, the film 108 is punctured by the punch pin 114 in response to pressing the deformable cap 110 into the deformed position, as shown in FIG. 3D. In response to puncturing the film 108 during step 204, and the pressure applied to the electrode gel 117 by pressing the deformable cap 110 into the deformed position, the electrode gel 117 is transferred from the storage location 116 into contact with the electrode 102. The electrode gel 117 is transferred from the storage location 116, which is located between the deformable cap 110 and the film 108, to the gel-retaining structure 106. Depending upon the embodiment, it may take several seconds for the gel-retaining structure 106 to become saturated with the electrode gel 117. Once the gel-retaining structure 106 has become saturated with the electrode gel 117, the electrode gel 117 comes into contact with the electrode 102.

At step 208, the clinician removes and discards the deformable cap 110 and the film 108 from the electrode module 100. At step 210, the clinician attaches the electrode module 100 to the skin of the patient. According to an exemplary embodiment, the clinician attaches the electrode module 100 to the skin of the patient on the side to which the film 108 was previously attached. According to an exemplary embodiment, the clinician may attach the electrode module 100 to the skin of the patient using the second adhesive 111 that is pre-applied between the film 108 and the conformable wall 104. As described previously, the electrode module 100 may also include a release coating applied between the film 108 and the conformable wall 104 and/or between the film 108 and the second adhesive 111 to facilitate the easy removal of the film 108 from the electrode module 100 during step 208. At step 212, if the sensor assembly includes additional electrode modules that need to be attached to the skin of the patient, then the method 150 returns to step 202. For example, according to the embodiment shown in FIG. 1, the sensor assembly 50 includes two electrode modules. As such, steps 202, 204, 206, 208, and 210 of the method 150 would need to be performed two times in order to attach both electrode modules 100 to the patient. According to the embodiment shown in FIG. 2, the sensor assembly 75 includes five electrode modules. As such, steps 202, 204, 206, 208, and 210 of the method 150 would need to be performed five times in order to attach all five electrode modules 100 to the patient. It should be appreciated by those skilled in the art that the method 150 may be used to attach sensor assemblies including any number of electrode modules to the patient. According to various embodiments, it may be necessary to connect the sensor assembly (50, 75) to a system before using the system and the sensor assembly (50, 75) to either detect electrical signals from the patient or provide electrical signals to the patient.

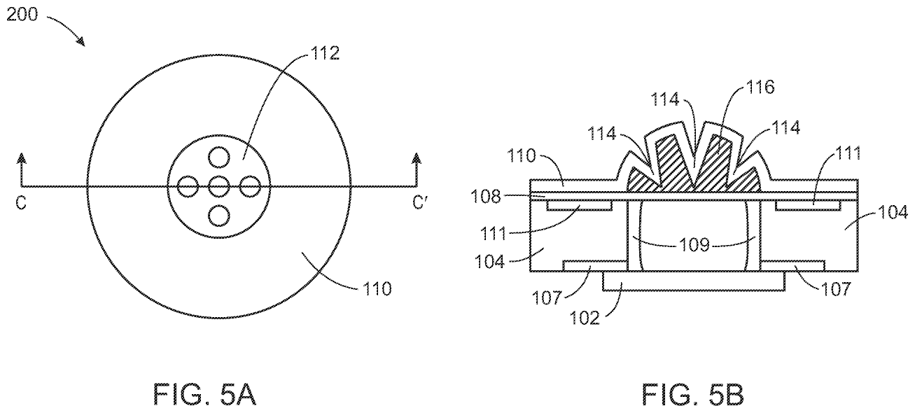

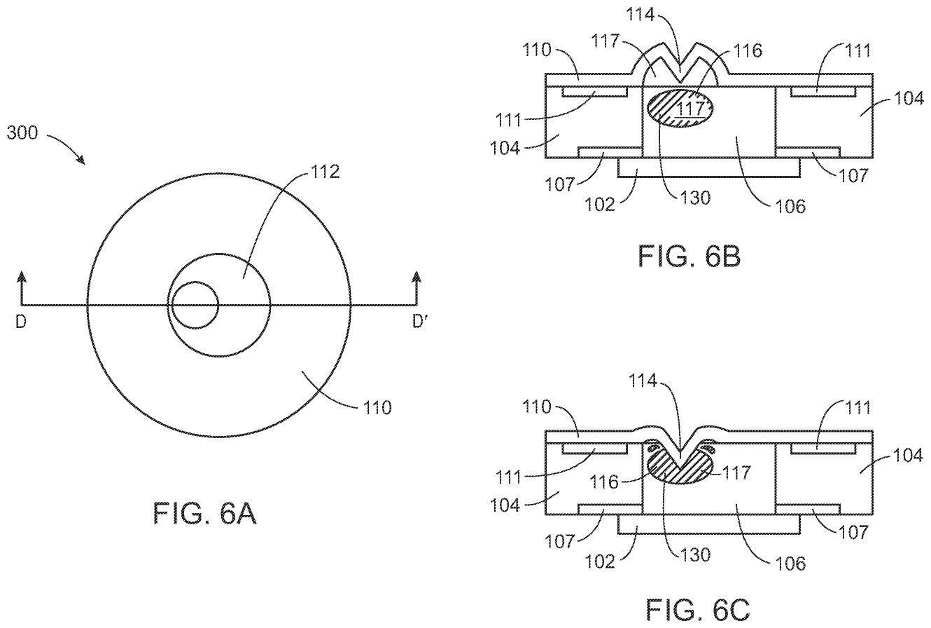

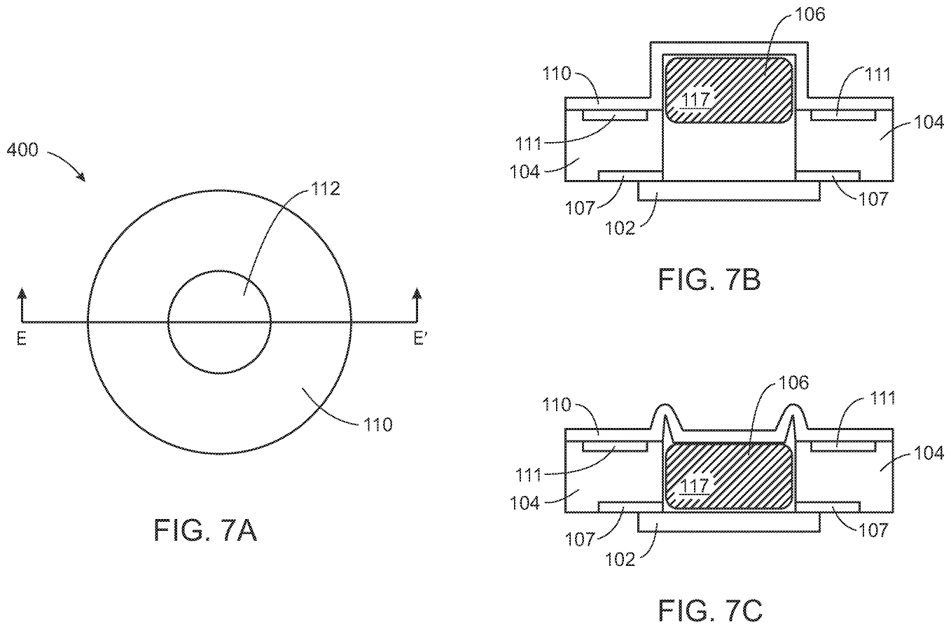

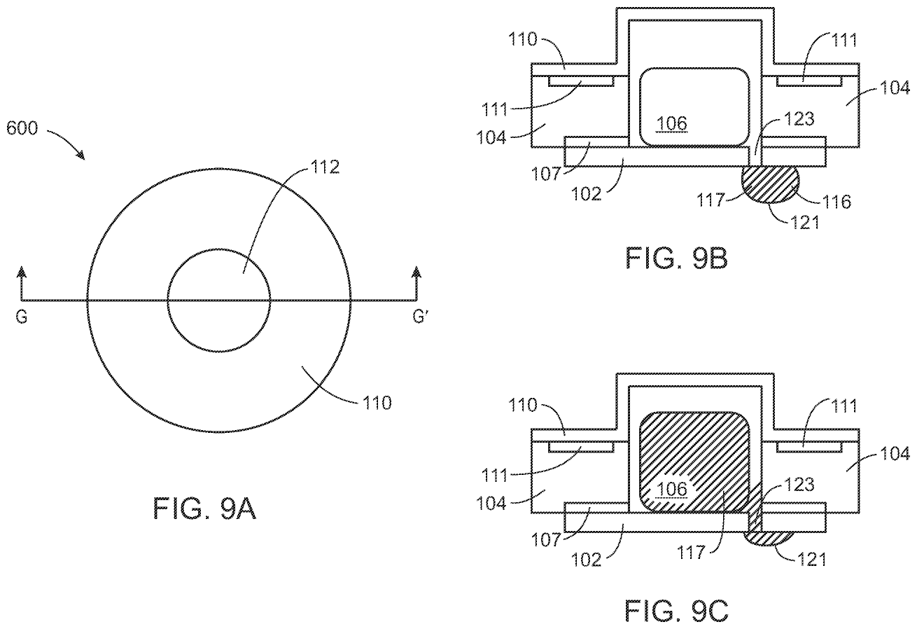

FIGS. 1 and 2 were described with respect to embodiments using electrode module 100, which was described in detail with respect to FIGS. 3A, 3B, 3C, and 3D. Various embodiments may use electrode modules which are different than the electrode module 100. Some exemplary electrode modules will be described according to various embodiments with respect to FIGS. 5A and 5B; FIGS. 6A, 6B, and 6C; FIGS. 7A, 7B, and 7C; FIGS. 8A, 8B, and 8C; and FIGS. 9A, 9B, and 9C. FIGS. 5A and 5B show an electrode module 200 according to an exemplary embodiment; FIGS. 6A, 6B, and 6C show an electrode module 300 according to an exemplary embodiment; FIGS. 7A, 7B, and 7C show an electrode module 400 according to an exemplary embodiment; FIGS. 8A, 8B, and 8C show an electrode module 500 according to an exemplary embodiment, and FIGS. 9A, 9B, and 9C show an electrode module 600 according to an exemplary embodiment. The electrode module 100, shown in FIGS. 1 and 2, may be replaced with an electrode module of a different configuration according to various embodiments. For example, the electrode module 100 shown in the sensor assembly 50 may be replaced with the electrode module 200, the electrode module 300, the electrode module 400, the electrode module 500, or the electrode module 600 according to various embodiments. Likewise, the electrode module 100 shown in the sensor assembly 75 may be replaced with the electrode module 200, the electrode module 300, the electrode module 400, the electrode module 500, or the electrode module 600 according to various embodiments. Additionally, the embodiments of the sensor assembly 50 or the sensor assembly 75 may use electrode modules of two or more different designs/configurations according to various embodiments. Additionally details about a non-limiting list of exemplary electrode modules will be provided hereinafter. Common reference numbers will be used to describe common components in the figures.

FIG. 5A illustrates a top view of an electrode module 200 of a sensor assembly in accordance with an embodiment and FIG. 5B illustrates a sectional view of the electrode module of FIG. 5A along line C-C′ in accordance with an embodiment. As discussed above, the electrode module 200 may replace one or both of the electrode modules 100 shown in the sensor assembly 50 of FIG. 1 and/or one or more of the electrode modules 100 shown in the sensor assembly 75 of FIG. 2 according to various embodiments. The deformable cap 110 of the embodiment shown in FIGS. 5A and 5B is shaped to define five punch pins instead of the single punch pin 114 shown in the embodiment represented in FIGS. 3A, 3B, 3C, and 3D. Three of the punch pins 114 are visible in the sectional view shown in FIG. 5B.

According to the embodiment shown in FIGS. 5A and 5B, the gel-retaining structure 106 is a deformable ring 109 configured to retain the electrode gel 117 in contact with the electrode 102. The deformable ring 109 may be constructed of a soft material such as rubber or silicone. The deformable ring 109 shown in FIG. 3B represents a single embodiment. Other embodiments may include a deformable ring of a different shapes to retain electrode gel 117.

The method 150 shown in FIG. 4 may be performed using an embodiment of the sensor assembly (50, 75) including the electrode module 200 described with respect to FIGS. 5A and 5B according to an embodiment. At step 204, one or more of the five punch pins 114 may puncture the film 108. Steps 202, 206, 208 and 210 of the method 200 would be substantially the same using the embodiment shown in FIGS. 5A and 5B as they were described with respect to the embodiment shown in FIGS. 3A, 3B, 3C, and 3D.

Other embodiments may use a different number of punch pins. For example, embodiments may use more than five punch pins or fewer than five punch pins. Additionally, the embodiments shown in FIGS. 3A, 3B, 3C, and 3D and in FIGS. 5A and 5B both rely on shaping the deformable cap 108 to form the punch pins. According to other embodiments, the punch pin/s may be formed using a different material than that which is used for the deformable cap 110. For example, one or more punch pins may be affixed to the deformable cap 110 in various embodiments.

FIG. 6A illustrates a top view of an electrode module 300 of a sensor assembly in accordance with an embodiment. FIG. 6B illustrates a sectional view of the electrode module 300 of FIG. 6A along line D-D′ in accordance with an embodiment. As discussed above the electrode module 300 may replace one or both of the electrode modules 100 shown in the sensor assembly 50 of FIG. 1 and/or one or more of the electrode modules 100 shown in the sensor assembly 75 of FIG. 2 according to various embodiments. FIG. 6C illustrates a sectional view of the electrode module of FIG. 6A along line D-D′ with the deformable cap 110 in a deformed position in accordance with an embodiment. The deformable cap 110 is in an undeformed position in FIG. 6B.

In the electrode module 300, the deformable cap 110 is shaped to define a single push pin 114 that is off-center with respect to the deformable cap 110. The electrode module 300 includes a capsule 130 disposed in the gel-retaining structure 106. The capsule 130 includes an outer skin containing electrode gel 117. The deformable cap 110 is shaped to define the push pin 114. The push pin 114 may, for example, be centered over the capsule 130. The sensor assembly 400 does not include a film such as the film 108 shown in FIGS. 3B, 3C, and 3D. The capsule 130 is filled with electrode gel 117. The outer skin of the capsule 130 is configured to be punctured by the punch pin 114 when the deformable cap 110 is in a deformed position. The second adhesive 111 is positioned between the deformable cap 110 and the conformable wall 104 in electrode module 300. According to an exemplary embodiment, the capsule 130 may be similar to a gel-capsule used for the oral delivery of drugs to a patient. The outer skin of the capsule 130 may, for example, be made from a plastic. According to various embodiments, the capsule 130 may be made from a thermoplastic, such as, for example, polystyrene, polyolefin, polyester, polyurethane, polyamide, polysulfone, polyethersulfone, polycarbonate, or the like.

FIG. 6C shows the deformable cap 110 in a deformed position. In the deformed position shown in FIG. 6C, the punch pin 114 has punctured the capsule 130, which allows the electrode gel 117 to flow from within the gel filled capsule 130 to the gel-retaining structure 106. The punch pin 114 is positioned off center with respect to the gel-retaining structure 106 in the embodiments shown in FIGS. 6A, 6B, and 6C to ensure that the outer skin of the capsule 130 does not block the entire electrode 102 after the capsule 130 has been punctured by the punch pin 114 as shown in FIG. 6C. While the embodiment shown in FIGS. 6A, 6B, and 6C has only a single punch pin and a single capsule, other embodiments may have one or both of a different number of capsules and/or a different number of punch pins. In other embodiments, the capsules may be distributed evenly throughout the gel-retaining structure 106, or the capsules may be biased towards certain regions of the gel-retaining structure 106 to ensure that the outer skins of the capsules do not interfere with the electrical contact between the electrode 102 and the patient after the electrode module 300 has been placed into contact with the skin of the patient.

The method 200 shown in FIG. 2 may be performed using the embodiment shown in FIGS. 6A, 6B, and 6C. Steps 202, and 210 of the method 200 would be substantially the same using the embodiment shown in FIGS. 6A, 6B, and 6C as they were described with respect to the embodiment shown in FIGS. 3A, 3B, 3C, and 3D. However, instead of performing step 204 which entails puncturing the film, the punch pin 114 would puncture the outer skin of the capsule 130 in response to pressure applied to the deformable cap 110. The storage location for the embodiment shown in FIGS. 6A, 6B, and 6C is within the capsule 130. Therefore, at step 206, the electrode gel 117 is transferred from the storage location 116 within the capsule 130 into the gel-retaining structure 106. According to an embodiment, pressure applied to the capsule 130 from the deformable cap 110 in the deformed position may help the electrode gel 117 to transfer from the capsule 130 to the gel-retaining structure 106. As described previously, FIG. 6C shows the deformable cap 110 in a deformed position. The punch pin 114 is shown breaking the outer skin of the capsule 130 in FIG. 6C, which allows the electrode gel 117 to flow from the capsule 130 to the gel-retaining structure 106. Once the gel-retaining structure 106 has become filled and/or saturated with electrode gel 117, steps 208 and 210 may be performed.

After puncturing the capsule 130, the deformable cap 110 is removed from the sensor assembly 300. The sensor assembly 300 does not have a film, so it is not necessary to remove a film before attaching the sensor assembly 300 to the skin of a patient at step 210.

FIG. 7A illustrates a top view of an electrode module 400 of a sensor assembly in accordance with an embodiment. FIG. 7B illustrates a sectional view of the electrode module 400 of FIG. 7A along line E-E′ in accordance with an embodiment. As discussed above the electrode module 400 may replace one or both of the electrode modules 100 shown in the sensor assembly 50 of FIG. 1 and/or one or more of the electrode modules 100 shown in the sensor assembly 75 of FIG. 2 according to various embodiments. FIG. 7C illustrates a sectional view of electrode module of FIG. 7A along line E-E′ with the deformable cap in a deformed position in accordance with an embodiment. In FIG. 7B, the electrode module 400 is depicted with the deformable cap 110 in an undeformed position.

In the embodiment of the electrode module shown in FIGS. 7A, 7B, and 7C, the gel-retaining structure 106 is saturated with electrode gel 117 with the deformable cap 110 in the undeformed position as shown in FIG. 7B. The gel-retaining structure 106 may be an open-cell sponge or a wire-frame sponge according to exemplary embodiments. The gel-retaining structure 106 is pre-saturated with electrode gel 117 prior to use. The gel-retaining structure 106 is in a first position near the deformable cap 110 as shown in FIG. 7B prior to use. When the clinician presses on the deformable cap 110, the gel-retaining structure 106 is translated into a second position where it is in contact with the electrode 102, as shown in FIG. 7C. Since the gel-retaining structure 106 is already saturated with electrode gel 117, the electrode gel 117 retained within the gel-retaining structure 106 is in contact with the electrode 102 when the gel-retaining structure 106 is in the second position as shown in FIG. 7C. After pressing on the deformable cap 110 and causing the gel-retaining structure 106 to translate from the first position to the second position, the clinician may remove the deformable cap 110 from the sensor assembly 400 and affix the sensor assembly 400 to the skin of a patient. Once the gel-retaining structure 106 is in contact with the electrode 102 and the sensor assembly 400 is attached to the skin of the patient, the electrode gel 117 retained within the gel-retaining structure 106 ensures a good electrical connection between the electrode 102 and the skin of the patient.

FIG. 8A illustrates a top view of an electrode module 500 of a sensor assembly in accordance with an embodiment. FIG. 8B illustrates a sectional view of the electrode module 500 of FIG. 8A along line F-F′ in accordance with an embodiment. FIG. 8C illustrates a sectional view of the electrode module 500 of FIG. 8A along line F-F′ in accordance with an embodiment. As discussed above, the electrode module 500 may replace one or both of the electrode modules 100 shown in the sensor assembly 50 of FIG. 1 and/or one or more of the electrode modules 100 shown in the sensor assembly 75 of FIG. 2 according to various embodiments. The deformable cap 110 is in an undeformed position in FIG. 8B. FIG. 8C is a sectional view of the electrode module of FIG. 8A with the deformable cap 110 in a deformed position. The film 108 in the embodiment shown in FIGS. 8A, 8B, and 8C is perforated with a plurality of perforations 113. Each of these perforations 113 is small enough to retain electrode gel within the storage location 116 defined by the deformable cap 110 and the film 108 when the deformable cap 110 is in the undeformed position.

However, when a clinician presses on the deformable cap 110, the electrode gel 117 exerts pressure on the film 108. This pressure causes the film 108 to stretch. The perforations 113 in the film 108 become enlarged, which allows the electrode gel 117 to pass through the perforations 113. According to other embodiments, the perforations 113 may not increase significantly in size in response to the pressure applied by the electrode gel 117. However, the increased pressure from the electrode gel 117 caused by the deformation of the deformable cap 110 may push the electrode gel 117 through the perforations 113. The deformation of the deformable cap 110 causes the electrode gel 117 to travel from the storage location 116 to the gel-retaining structure 106. Once enough electrode gel 117 moves to the gel-retaining structure 106 to make contact with the electrode 102, the clinician may remove the deformable cap 110 and the film 108 and affix the sensor assembly 600 to the skin of the patient.

FIG. 9A illustrates an electrode module 600 of a sensor assembly in accordance with an embodiment. FIG. 9B illustrates a sectional view along the line G-G′ shown in FIG. 9A. FIG. 9C illustrates a sectional view along the line G-G′ shown in FIG. 9A. As discussed above the electrode module 600 may replace one or both of the electrode modules 100 shown in the sensor assembly 50 of FIG. 1 and/or one or more of the electrode modules 100 shown in the sensor assembly 75 of FIG. 2 according to various embodiments. The embodiment shown in FIGS. 9A, 9B, and 9C includes a deformable button 121 that that defines a storage location 116 for electrode gel 117. The deformable button 121 may be formed of any flexible material such as rubber, or a plastic, such as, for example, polystyrene, polyolefin, polyester, polyurethane, polyamide, polysulfone, polyethersulfone, polycarbonate, or the like. The storage location 116 is filled with electrode gel 117 according to an embodiment. FIG. 9B shows the deformable button 121 in an undeformed state and FIG. 9C shows the deformable button 121 is a deformed state. According to an embodiment, a clinician may first remove the deformable cap 110 from the electrode module 600 and affix the electrode module 600 to the skin of a patient. For example, the electrode module 600 may be affixed to the patient by the second adhesive 111 after the deformable cap 110 has been removed from the electrode module 600. Other embodiments may include a non-deformable cap in place of the deformable cap 110 show in FIGS. 9A, 9B, and 9C. After affixing the electrode module 600 to the patient, the clinician may apply pressure to the deformable button 121 in order to cause the deformable button 121 to transition from the undeformed state shown in FIG. 9B to the deformed state shown in FIG. 9C. Applying pressure to the deformable button 121 applies pressure on the electrode gel 117 (depicted by the hatching) within the deformable button 121. The pressure on the electrode gel 117 causes the electrode gel to travel through a via 123 and into contact with the gel-retaining structure 106. The electrode gel 117 is transferred from the storage location 116 withing the deformable button 121 to the gel-retaining structure 106. The electrode gel 117 in the gel-retaining structure ensures a good electrical connection between the electrode 102 and the skin of the patient to which the electrode module 600 is affixed. The design of electrode module 600 advantageously allows for the electrode module 600 to be attached to the skin of the patient before the electrode gel 117 is transferred from the storage location 116 into contact with the electrode 102. This ensures that the electrode gel 117 does not interfere with the attachment of the electrode module 600 to the skin. That is, by attaching the electrode module 600 to the skin of the patient before applying pressure to the deformable button 121 and transferring electrode gel 117 from the storage location 116 to the gel-retaining structure 106, it is impossible for any of the electrode gel 117 to interfere with the second adhesive 111 located between the deformable cap 110 and the conformable wall 104 that is used to secure the electrode module 600 to the skin of the patient.

While only explicitly shown in FIG. 3C, those skilled in the art should appreciate that the conformable wall 104 of other embodiments, such as the electrode module 200, the electrode module 300, the electrode module 300, the electrode module 400, the electrode module 500, and the electrode module 600 may also be shaped to define a passageway similar to the passageway 118 shown with respect to the electrode module 100 in order to permit the evacuation of air as electrode gel 117 is moving into the gel-retaining structure 106.

The embodiments described hereinabove all allow for the sensor assemblies to be stored for much longer periods of time than conventional solutions since the electrode gel 117 is not in contact with the electrode while the sensor assembly (including one or more electrode modules) is being stored prior to use. By separating the electrode gel 117 from the electrode 102, the electrode gel 117 does not cause any degradation of the electrode 102. This, in turn, enables to the sensor assembly to have a much longer shelf-life than conventional solutions, while still providing the ease-of-use by having the electrode gel 117 integrated into each electrode module.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions and types of materials described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc., are used merely as labels, and are not intended to impose numerical requirements on their objects. Further, the limitations of the following claims are not written in means-plus-function format and are not intended to be interpreted based on 35 U.S.C. § 112(f), unless and until such claim limitations expressly use the phrase “means for” followed by a statement of function void of further structure.

This written description uses examples to disclose the invention, including the best mode, and also to enable any person skilled in the art to practice the invention, including making and using any sensor assemblies or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A sensor assembly configured to be attached to a patient for at least one of detecting an electrical signal from the patient or providing an electrical signal to the patient, wherein the sensor assembly comprises one or more electrode modules, wherein each of the one or more electrode modules comprises:
an electrode;
electrode gel disposed in a storage location on the electrode module, wherein the electrode gel is not in contact with the electrode in the storage location; and
a deformable cap configured to move the electrode gel from the storage location into contact with the electrode in response to a deformation of the deformable cap.

2. The sensor assembly of claim 1, wherein each of the one or more electrode modules further comprises a gel-retaining structure configured to retain the electrode gel in contact with the electrode after the electrode gel has moved from the storage location into contact with the electrode.

3. The sensor assembly of claim 1, wherein each of the one or more electrode modules further comprises a film disposed between the deformable cap and the electrode, wherein the storage location is a chamber defined by the deformable cap and the film.

4. The sensor assembly of claim 3, wherein each of the one or more electrode modules further comprises a gel-retaining structure configured to retain the electrode gel in contact with the electrode after the electrode gel has moved from the storage location into contact with the electrode.

5. The sensor assembly of claim 4, wherein the gel-retaining structure is an open-cell sponge.

6. The sensor assembly of claim 4, wherein the gel-retaining structure is a wire-frame sponge.

7. The sensor assembly of claim 4, wherein the gel-retaining structure is a deformable ring configured to retain the electrode gel within a center region defined by the deformable ring.

8. The sensor assembly of claim 4, wherein the deformable cap includes at least one punch pin configured to puncture the film in response to the deformation of the deformable cap.

9. The sensor assembly of claim 4, wherein the film is perforated with a plurality of perforations, wherein the plurality of perforations are sized to allow the passage of the electrode gel from the storage location into contact with the electrode in response to pressure exerted on the film from the electrode gel in response to the deformation of the deformable cap.

10. The sensor assembly of claim 1, wherein each of the one or more electrode modules further comprises a gel-retaining structure configured to retain the electrode gel in contact with the electrode after the electrode gel has moved in response to the deformation of the deformable cap, and wherein the storage location comprises a capsule disposed within the gel-retaining structure, and wherein the deformable cap is configured to burst the capsule when in a deformed position.

11. The sensor assembly of claim 1, wherein each of the one or more electrode modules further comprises a gel-retaining structure that is saturated with the electrode gel, wherein the deformable cap is configured to move the saturated gel-retaining structure from a first position to a second position in response to the deformation of the deformable cap, wherein the electrode gel is not in contact with the electrode when the saturated gel-retaining structure is in the first position and wherein the electrode gel is in contact with the electrode when the saturated gel-retaining structure is in the second position.

12. The sensor assembly of claim 1, wherein the deformable cap is configured to be removed from the electrode module after it has been deformed.

13. A method of using a sensor assembly including one or more electrode modules, wherein each of the one or more electrode modules comprises an electrode and a deformable cap, the method comprising:
pressing the deformable cap into a deformed position; and
transferring electrode gel from a storage location into contact with the electrode in response to said pressing the deformable cap.

14. The method of claim 13, wherein each of the one or more electrode modules comprises a film attached to the deformable cap, wherein the deformable cap comprises at least one punch pin, wherein said pressing the deformable cap into the deformed position causes the at least one punch pin to puncture the film and allows the electrode gel to flow through the punctured film into contact with the electrode.

15. The method of claim 13, wherein each of the one or more electrode modules comprises a film with a plurality of perforations, wherein pressing the deformable cap into the deformed position exerts pressure on the film and allows the electrode gel to flow through at least some of the plurality of perforations.

16. The method of claim 13, further comprising removing the deformable cap after said pressing a deformable cap on the sensor assembly into a deformed position, and attaching the sensor assembly to a skin surface of a patient after said removing the deformable cap.

17. The method of claim 13, further comprising attaching the sensor assembly to a skin surface of a patient before said pressing the deformable cap.

18. A sensor assembly comprising:
an electrode;
a gel or liquid retention structure in contact with the electrode;
a gel or liquid contained within a storage location, wherein the gel or liquid is not in contact with the electrode while in the storage location; and
a deformable cap configured to move the gel or liquid from the storage location into the gel or liquid retention structure in contact with the electrode in response to a deformation of the deformable cap.

19. The sensor assembly of claim 18, further comprising a film disposed between the storage location and the gel or liquid retention structure.

20. The sensor assembly of claim 19, wherein the deformable cap comprises at least one punch pin configured to puncture the film in response to the deformation of the deformable cap, wherein the deformation of the deformable cap is configured to move the gel or liquid from the storage location into the gel or liquid retention structure.
